(19) United States
(12) Patent Application Publication
(10) Pub. No.: US 2012/0053450 A1
(43) Pub. Date: Mar. 1, 2012

(54) Trans-perineal prostate MR elastography

(76) Inventors: Septimiu Salcudean, Vancouver (CA); Ramin S. Sahebjavaher, West Vancouver (CA); Ralph Sinkus, Parmain (FR)

(21) Appl. No.: 13/104,081

(22) Filed: May 10, 2011

Related U.S. Application Data
(60) Provisional application No. 61/395,058, filed on May 10, 2010.

(51) Int. Cl. A61B 5/055

(52) U.S. Cl. 600/421

(57) ABSTRACT

The present invention relates to a method and apparatus for imaging the mechanical properties of the prostate of a patient non-invasively. The apparatus generally comprises a magnetic resonance scanner, a vibration assembly coupled to the perineal region of the patient, and a driver that drives the mechanical exciter. The method generally comprises positioning the vibration assembly against the perineal region of the patient, vibrating the mechanical exciter to cause deformational excitation of a tissue region contacted in the perineum, capturing a series of images in time (snapshots) of the tissue region using the MR scanner, and finally processing the displacement images to generate maps of mechanical properties of images tissue.

This application claims the benefit of U.S. Provisional Patent Application No. 61/395,058, filed on May 10, 2010, which is incorporated herein by reference.

### FIELD OF THE INVENTION

The present invention relates to diagnostic imaging generally, and measurement of the mechanical properties of the prostate using magnetic resonance elastography specifically.

### BACKGROUND OF THE INVENTION

The basis of medical imaging is the measurement of a property of tissue that varies with tissue composition. Medical images are formed by displaying intensities as a function of these properties measured at various locations in the body. Mechanical properties of tissue are important indicators of disease potential. Indeed, palpation techniques are commonly used by medical doctors to determine the potential for disease, for example, stiffer tissue regions that can be felt as harder objects can indicate the presence of cancer. This is the basis for a number of clinical examinations such as the digital rectal examination for prostate cancer.

Magnetic Resonance Elastography (MRE) is a medical imaging modality that aims to depict elasticity, a mechanical property of tissue. Elasticity is also referred to as stiffness, or the inverse compliance. For this imaging technique, a mechanical excitation is applied in the proximity of the tissue of interest (e.g., prostate) and the resulting deformation is measured. The resulting deformation image is referred to as a strain image and is post-processed to extract information such as viscoelastic properties (e.g., shear modulus and viscosity). These mechanical properties are then displayed as a map of stiffness (or other meaningful mechanical properties) of the imaged object.

Clinical uses of elastography were first demonstrated in the field of ultrasound as described in U.S. Pat. No. 5,107,837 by Ophir et. al. Entitled “Method and Apparatus for Measurement and Imaging of Tissue Compressibility and Compliance.” Shortly afterwards elastography was introduced in the field of magnetic resonance imaging (MRI) by Ehman and Muthupillai as described in U.S. Pat. No. 5,825,186 entitled “Method for Producing Stiffness-Weighted MR Images” and U.S. Pat. No. 5,977,770 by Ehman entitled “MR Imaging of Synchronous Spin Motion and Strain Waves.” In the following years elastography was shown to be of clinical value for the detection and staging of hepatic (liver) fibrosis by Sinkus et. al. “Liver fibrosis: non-invasive assessment with MR elastography” in the Journal NMR in Biomedicine 2006, pages 173-179, and Ehman et. al. “Assessment of Hepatic Fibrosis With Magnetic Resonance Elastography” in the Journal of Clinical Gastroenterology and Hepatology, volume 5, Issue 10, October 2007, pages 1207-1213. Elastography imaging of the breast has been successfully demonstrated and published by Sinkus et. al. in “Viscoelastic shear properties of in vivo breast lesions measured by MR elastography” in the Journal of Magnetic Resonance Imaging volume 23, 2005, pages 159-165. Elastography of the brain is also published by Papazoglou and Braun et. al. in “Three-dimensional analysis of shear wave propagation observed by in vivo magnetic resonance elastography of the brain” in Acta Biomaterialia, volume 3, 2007, pages 127-137. More recently, elastography of the lung was demonstrated by Ehman et. al. In U.S. Pat. No. 2006/0264736 entitled “Imaging Elastic Properties of the Lung with Magnetic Resonance Elastography.” MRE of the prostate ex-vivo was demonstrated first by Dresner, Rossman and Ehman, published in the Proceedings of the International Society for Magnetic Resonance in Medicine entitled “MR Elastography of the Prostate” in 1999. MRE of the prostate in-vivo was demonstrated by Sinkus et. al. and published in “In-Vivo Prostate Elastography”, Proceedings of International Society of Magnetic Resonance in Medicine, volume 11, page 586, 2003.

Elastography is closely related to the estimation of deformation in the presence of an externally applied mechanical stress, or force. In dynamic elastography, an external periodic mechanical excitation is applied to the tissue of interest by an actuator. Actuators that are used for MRE must be compatible with the MR environment, or in other words, they must be immune to strong static magnetic fields and fast-switching large-amplitude gradient fields. In the literature there are two types of mechanical excitation devices (or actuators) that may be suitable (or are currently used) for inducing mechanical deformation in the prostate in an MRI scanner, namely invasive or non-invasive. Non-invasive actuators are applied over the surface of the skin close to the tissue of interest. For prostate elastography the actuators are typically applied on or close to the pubic bone (or belly); they may also be applied on the bottom end of the spine (or the back) of the patient. Several non-invasive actuators have been developed as described in U.S. Pat. No. 5,952,828, 2010/0005892, U.S. Pat. No. 7,034,534 and the publications referred to above and also by Kemper, Sinkus et. al. “MR Elastography of the Prostate: Initial In-vivo Application.” published in Fortschritte auf dem Gebiete der Röntgenstrahlen and der Nuklearmedizin (Advances in the area of X-ray and Nuclear Medicine), volume 176, pages 1094-1099, 2004. Invasive actuators, on the other hand, contain at least one component that penetrates the surface of the skin or enters a natural orifice of the body. These actuators are either applied through the rectum or the urethra as described in U.S Pat. No. and 2009/0209847, 2010/0045289 and the following publication Plewes et. al. “In Vivo MR Elastography of the Prostate Gland Using a Transurethral Actuator” Magnetic Resonance in Medicine, volume 62, 2009, pages 665-671. Alternatively, the mechanical excitation is applied to a needle that penetrates the skin as described in U.S. Pat. No. 2008/0255444.

A magnetic resonance elastography system with hydraulic driver has been described by Li in US Patent Application Publication number 20090209847, the system comprising a hydraulic driver including a pump, a hydraulic piston-cylinder unit coupled to the pump, and a tube assembly with a proximal end coupled with the piston-cylinder unit, and a distal end in fluid communication to a passive actuator. The transmission system from Li US at Appl 20090209847 is not pressurized, i.e. the fluid transmission is not naturally compressed by a compliant spring. Therefore, when the passive actuator is placed against the body in order to generate vibrations, a significant pre-load against the subject's tissue must exist in order for the rapid accelerations both towards and away from the image tissue to be accommodated. Basically, the acceleration of the passive actuator is away from the tissue being compressed, this acceleration is limited by the force generated by the atmospheric pressure. Furthermore, Li US Pat Appl 20090209847 also does not describe a means of connecting and disconnecting the transmission components in order to enable the connection of multiple passive actuators to the same hydraulic driver.

Inducing mechanical deformation non-invasively into the abdominal region for elastography imaging is challenging in particular in the prostatic tissue. This is because the prostate is surrounded by the pelvis (i.e., bone) and many body structures (e.g., ligaments and fatty tissue) that scatter and dampen the mechanical waves and in essence prevent the mechanical waves from entering the region of interest. As a result, applying mechanical excitations to the pubic bone does not guarantee large mechanical shear waves in the prostate, regardless of the position of the patient (prone or supine). This has been tested and observed by the inventors and in Sinkus et. al. “In-Vivo Prostate Elastography”, Proceedings of International Society of Magnetic Resonance in Medicine, volume 11, 2003 page 586.

For conventional MRE the mechanical properties are estimated based on mechanical shear waves. However, shear waves are easily damped and may not travel deep enough into tissue. This is especially true for prostatic tissue that is surrounded by various tissues and bone structures. To ensure sufficient penetration of mechanical waves, longitudinal waves are applied to the surface of the tissue. Longitudinal waves travel faster and are not damped as much as shear waves. When longitudinal waves encounter a tissue interface (i.e., travel from one tissue type to the next) they are transformed into shear waves by mode conversion (Sinkus and Tanter et. al. “Viscoelastic shear properties of in vivo breast lesions measured by MR elastography” journal of Magnetic Resonance Imaging, volume 23, 2005 pages 159-165). Using this technique both mechanical shear and longitudinal waves are present in the tissue of interest. The resulting displacements that are recorded by the MRE imaging sequence contain both components of the mechanical waves (longitudinal and shear), which are separated and processed as described in U.S. Pat. No. 2009/0124901.

### BRIEF SUMMARY OF THE INVENTION

This patent application describes a technique that was successfully applied to induce large amplitude mechanical waves into the prostate by applying the mechanical excitation apparatus to the perineal region of the patient.

To generate mechanical shear waves in the prostate with a large enough amplitude, this invention proposes that vibrations be applied at the perineum via a coupling surface that is attached to a mechanical exciter. In a preferred embodiment a coupling surface of the mechanical exciter undergoes a reciprocal motion that generates longitudinal waves against the perineum that travel effectively to the prostate. This excitation is non-invasive, as only the surface of the perineum is touched. Furthermore, this excitation can be applied over the undergarment of the patient and can therefore be non-invasive.

According to one aspect of the invention, there is provided a method for applying vibrations for generating a magnetic resonance elastography scan of a subject comprising, positioning the end-effector of a mechanical exciter against the perineum of said subject and applying mechanical vibrations generated by said mechanical exciter to said end-effector.

According to another aspect of the invention, there is provided a method for applying vibrations for generating a magnetic resonance elastography scan of a subject comprising, positioning the end-effector of a mechanical exciter against the perineum of said subject, inspecting a survey scan image which includes both the prostate and the end-effector, repositioning said end-effector against the perineum based on inspection of said image, and applying mechanical vibrations generated by said mechanical exciter to said end-effector.

In various embodiments of the invention, the mechanical vibrations can be generated by MRI compatible actuators such as piezoelectric actuators, or by voice coil actuators that use the magnetic field of the scanner to generate forces when driven by currents. These function in the scanner room or even use the strong magnetic fields within it. Alternatively, the mechanical vibrations may be generated remotely from the scanner coil, and these mechanical vibrations may be transmitted to the end-effector against the patient's body by a hydraulic or pneumatic transmission, or by other transmissions using cables, tendons or rods.

According to one aspect of the invention, there is provided an apparatus for imaging the mechanical properties of a tissue region of a patient in a non-invasive manner, the apparatus comprising a mechanical exciter, means to adjustably couple said mechanical exciter to the perineum of a patient, and means to adjustably couple said mechanical exciter to the scanning table.

In terms of the mechanical excitation apparatus, a preferred embodiment of the vibration source, or source, in short, may be an electromagnetic actuator that is placed away from the MRI scanner while the mechanical motion is transmitted, via a transmission medium or “transmission”, e.g. a hydraulic transmission, to the perineal region of the patient, by an end-effector.

In the case of MRE, the actuator and its accessories need to be MR safe and not interfere with the imaging hardware by producing imaging artefacts.

The end-effector may be designed such as to allow the use of endo-rectal coils for acquiring higher resolution MRI and MRE images.

A static pressure must be kept on the coupling surface so that it maintains proper contact with the perineum. If the contact between the tissue and mechanical exciter is compromised the mechanical waves induced in the tissue will be different from what was intended resulting in inconsistent outcomes. A sensor may be incorporated in the driver system to detect if a proper contact exists between the patient and the driver.

A sensor may be implemented in the end-effector to monitor the mechanical response which may be used to compare to the desired signal. The signal may also be used in a closed loop system to correct for any drifts from the desired waveform.

A positioning apparatus is described that is used to align the mechanical driver with respect to the patient.

### BRIEF DESCRIPTION OF THE DRAWINGS

FIG. 1 is a side (sagittal) view of the mechanical setup of the elastography imaging apparatus for the measurement of the mechanical properties of prostate tissue in a patient using a trans-perineal transducer, according to one embodiment of the invention.

FIG. 2 shows the magnetic resonance elastography image acquisition system with the patient and passive end of the exciter inside a conventional MRI machine. The active end of the exciter is located in the console room that is behind the radiofrequency shielded room.

FIG. 3 is a cross-sectional representation of the passive and active end of the hydraulic exciters shown in FIGS. 1 and 2. The passive end is coupled to the perineal area of the patient.

FIG. 4 is a schematic perspective and exploded view of the current embodiment of the positioning apparatus of the passive exciter that is located in a MRI scanner.

FIG. 5 is a schematic perspective view of the current and some alternate embodiments of the endpoint attachments of the passive exciter shown in FIGS. 3 and 4.

FIG. 6 is a schematic showing the timing and signal flow from the MR console to the driver, mechanical transducer and the tissue of interest.

FIG. 7 is a series of conventional and elastography images acquired by one embodiment of this invention as shown in FIGS. 1 and 2, where the amplitude of the mechanical waves are clearly detectable in the prostate tissue, demonstrating the feasibility and effectiveness of the trans-perineal excitation method.

FIG. 8 shows alternate embodiments of the positioning mechanisms.

FIG. 9 shows a mechanism for quick connection and disconnection of two already-pressurized hydraulic pipes.

FIG. 10 shows the T2-weighted sagittal image of a subject's prostate, indicating the location of the actuator against the perineum.

### DETAILED DESCRIPTION OF EMBODIMENTS OF THE INVENTION

The embodiments described herein relate to a method and apparatus of a magnetic resonance elastography (MRE) image acquisition apparatus for the measurement of the mechanical properties of a tissue region in a patient using a mechanical transducer. Particularly, the embodiments relate to inducing or transferring mechanical shear and longitudinal vibrations into the lower abdomen, specifically in the prostate area for male patients. This is achieved by applying a mechanical exciter to the perineal region of the patient that generates mechanical longitudinal vibrations.

### Anatomy, Positioning of Exciter at Patient End

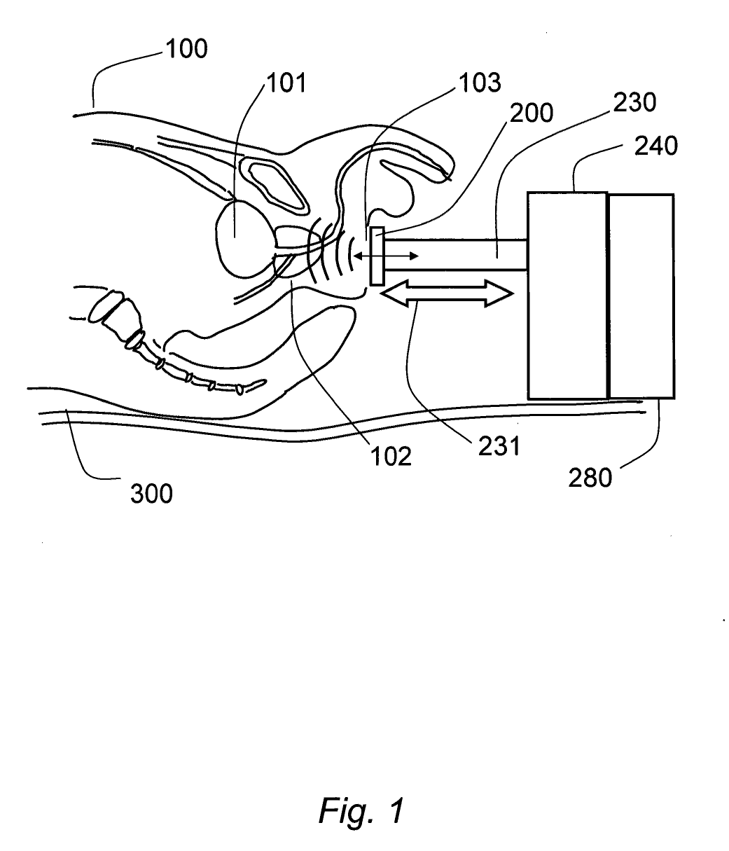

FIG. 1 shows the abdominal section of a patient lying in a typical MR scanner being prepared for a MRE scan. The patient 100 rests in the supine position (facing up) on the table of the magnetic resonance imaging (MRI) scanner 300. The patient is positioned such that the feet enter the MR magnet (feet-first) while the head stays out. In other possible embodiments the patient may be positioned in the prone position (facing down) and/or head first depending on other MR examinations that may precede or follow the MRE examination. For example, if an endo-rectal radiofrequency (RF) coil is planned to be used for a MRE scan or other MRI scan it may be best if the patient is in supine and head-first position. The exciter 240 that is connected to the positioning mechanism 500 also rests on the MRI table and between the legs of the patient. The exciter 240 can be positioned with respect to the patient on an individual basis. The vibrations of the exciter are transferred longitudinally 231 to the patient via a transmission element 230 which may also include an end-point attachment having a coupling surface 200, also referred to as the endpoint attachment or end-effector. In one embodiment of the invention, the end-effector 200 is shaped such that it fits the shape of the perineal area 103 and maintains contact at all times during the MRE scan. Also, the shape and location of the end-effector may be designed such that they allow the use of an endo-rectal RF coil if a higher resolution MRE scan or a different abdominal MR scan is desired. The mechanical vibrations are applied in the horizontal direction as in 231 to generate longitudinal mechanical waves in the perineum 103 that effectively propagate to the prostate 102.

### Positioning of Patient Relative to Exciter and MRI Scanner

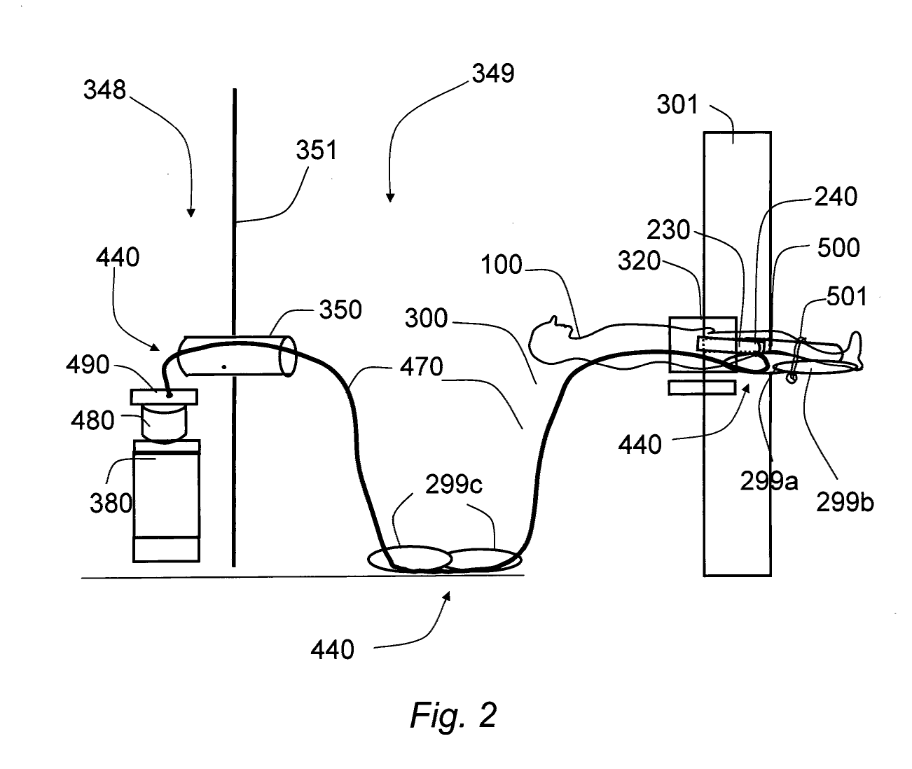

Referring to FIG. 2, in the current embodiment the actuator is pressed against the perineum region 103 of the patient via the positioning mechanism 500. The positioning mechanism is placed on standard MRI safe sandbags 299a (proximal) and 299b (distal), and is locked in place by a strap 501 that is connected to slots in the MR table 300. The static pressure between the patient 100 and the coupling surface 200 is in the range of 1-20 kPa. The angle 520 between the patient (or MR table) and the long axis of the exciter 240 is typically in the range of −45 to 45 degrees and is determined based on the anatomy of the patient by the technician. A phased array cardiac (or abdominal) RF coil 320 is strapped around the abdomen of the patient for imaging purposes, and is adjusted in the cranio-caudal direction to capture the prostate gland by the technician. An endo-rectal coil can also be used if higher resolution scan is necessary. The endo-rectal coil does not interfere with the placement of the end-effector 200 against the perineum 203, because as it lies anteriorly and inferiorly relative to the endo-rectal coil. Once the patient is positioned with respect to the exciter 240, the MR table 300 is moved into the bore scanner 301 until the anatomy of interest is at the iso-center of the magnet. Variations of the above, e.g. exciter pressure, coil positioning, patient location for scanning, are possible and obvious to implement for one skilled in the art.

### Hydraulic Exciter (Current Embodiment)

In one embodiment, the mechanical exciter is of a hydraulic type with a passive end 240 coupled to the patient 100 and an active end 440 that is connected to a mechanical vibration source 480. The two ends of the hydraulic exciter are connected together via a semi-rigid tube 470 that passes from the MRI scanner room 349 through the RF shield 351 of the MRI room via a waveguide 350 to the console room 348. To suppress vibration modes in the pipe 470, multiple sand bags 299c can be used to press the pipe 470 to the floor of the scanner room 349. Alternatively, part of the tubing can be made of rigid pipes to increase the overall stiffness of the transmission for higher frequency response and fewer problems with resonance modes. Thus only one end of the pipe needs to be of semi-rigid type material so that it can be positioned easily against the patient.

### Alternate Embodiments of the Exciter

In alternate embodiments, the mechanical exciter may be of other types. The main point is to generate longitudinal mechanical excitation (vibration) at the surface of the perineum with a transducer that does not interfere with the operation of a conventional magnetic resonance imaging machines. For example, it may be of electromechanical type that either uses its own shielded permanent magnet or utilizes the magnetic field of the MR scanner itself (such as U.S. Pat. Nos. 6,037,774 or 5,952,828). Usually the problem with these actuators is that they introduce artefacts in the images if they are positioned close to the magnet as they distort the homogeneity of the magnetic field. Another problem with electromagnetic exciters is that it is hard to position them because they need to be positioned with respect the magnetic field of the MRI magnet. Another possibility is to use piezoelectric actuators that have superb frequency response but have limited amplitude (U.S. Pat. No. 2008/0255444). Pneumatic solutions (U.S. Pat. No. 7,034,534) are also possible which have advantages similar to hydraulic actuators but may have problems with stability of the phase and typically they also require larger diameter pipes.

In general, actuators for MRE are made of two components: source and transmission. Usually the source is either electromagnetic or piezoelectric which generates the desired mechanical excitation waveform. The transmission element is how the vibration from the source is transferred to the region of interest. One way this can be achieved is by a mechanical coupling, such as a rod that rests on skin or a needle that penetrates the tissue, which directly couples the source to the region of interest. Alternatively, a pneumatic (or acoustic) solution can be used where the mechanical waves are transferred by sound waves. Another possibility is to use hydraulic transmission which uses a hydraulic fluid to transfer the mechanical vibration to the region of interest. This is summarized in the table below. The actuator described in the current embodiment of this patent application has an electromagnetic source with a hydraulic transmission.

| SOURCE | TRANSMISSION |
|---|---|
| Electromagnetic | Mechanical coupling |
| B₀ | Push rod |
| Permanent magnet (Speaker, Motor) | Needle |
| Electromagnet | Hydraulic |
| Piezoelectric | Pneumatic |
| Stack (Linear motion) | |
| Strip (Bending) | |

### Hydraulic Fluid

The fluid inside the pipe 470 should be non-compressible and may be chosen such that it lacks a MR signal. In the current prototype, tap water was used with no adverse effect on the MRI or MRE images. To suppress the MR signal from the water insider the pipe, ions (such as Cu²⁺, from cupper sulphates CuSO₄) can be added to reduce the T₂ to times shorter than the echo time of the imaging sequence. This way the signal from the water inside the pipe dies away before the signal from the tissue of interest is acquired.

### Timing and Sequence

In conventional dynamic MR elastography, harmonic and synchronous mechanical waves are imaged by applying bipolar gradient fields in the direction in which the displacements are encoded as described in U.S. Pat. Nos. 5,977,770; 5,825,186; 7,025,253; 2009/0124901. Conventionally, the frequency of the bipolar gradient is set to be the same frequency as the mechanical excitation. This has limitations in term of the echo time TE that results in lower Signal-to-Noise ratio (SNR) that may affect the elastography images. To overcome this, a second-harmonic imaging mode is utilized to shorten the echo time in order to produce better MRE images. In this technique the frequency of the bipolar gradients is set to twice the frequency of the mechanical excitation as thoroughly explained by Sinkus et. al. “Magnetic Resonance Elastography in the Liver at 3 Tesla Using a Second Harmonic Approach” published in the journal of Magnetic Resonance in Medicine, volume 62, in 2009, pages 284-291.

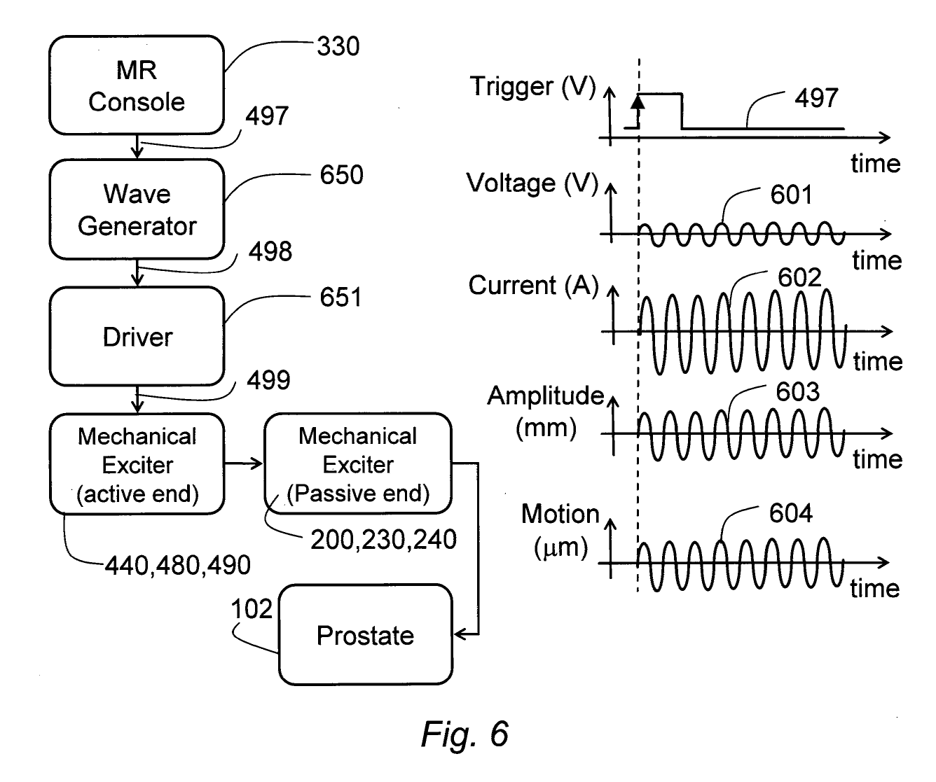

Referring to FIG. 6, the vibration source 480 is connected to a power amplifier driver 651 that amplifies the electrical signal 602 coming from the arbitrary waveform generator (AWG) 601. Typically a sinusoidal waveform is used for the mechanical excitation in conventional MRE as shown in 494. The parameters of the excitation signal (e.g. frequency) are determined by the pulse sequence used for the MRE exam. The mechanical excitation is synchronized with the pulse sequence of the MRE exam. To do this, the console 330 of the MR system generates a trigger pulse 600 that is an input to the arbitrary waveform generator 650 that starts the pre-programmed waveform (e.g., sine wave). The mechanical excitation is generated by an electromagnetic exciter 480 and is transferred to the active end of the hydraulic exciter 440 via an attachment mechanism 490. The mechanical excitation wave passes through the tube 470 to the passive end of the actuator 240. The vibration from the exciter 240 travels to the perineal region 103 via the transmission element 230 and the end point attachment 200. The vibration waveform at the perineal area is shown in FIG. 6 in 603. The amplitude of the mechanical wave inside the tissue located deeper in the body will be much lower as represented in 604. The mechanical vibration at one point in the tissue is of the same shape as 601 (i.e., sinusoidal) but may be shifted in time (phase) depending on the location of the point of interest and the speed of the wave in that particular tissue.

### MRE Exam, Imaging Settings

A typical MR exam includes performing (i) a reference scan to determine the intensity maps of the RF coil (e.g., cardiac coil), (ii) a survey scan to locate the tissue of interest (e.g., prostate) and (iii) a specialized higher resolution imaging protocol suitable for the tissue of interest. These imaging protocols are selected from a set of standard protocols known to those skilled in the art and available from the MR scanner itself. For an MRE scan, once the patient and the mechanical exciter are positioned a standard MR exam is performed. Following the MRI exam, the MRE scan starts by selecting the imaging parameters for an elastography based image. In the current embodiment, in addition to the standard imaging parameters the frequency and amplitude of excitation may be also changed. Then, the vibration is enabled and the sequence is run. In a current embodiment the imaging parameters for the MRE exam consist of imaging a stack of seven consecutive axial slices of the prostate with isotropic voxel size of 1.5 mm. An echo-planar imaging (EPI) readout with and EPI acceleration factor of three was chosen.

### Sample Images

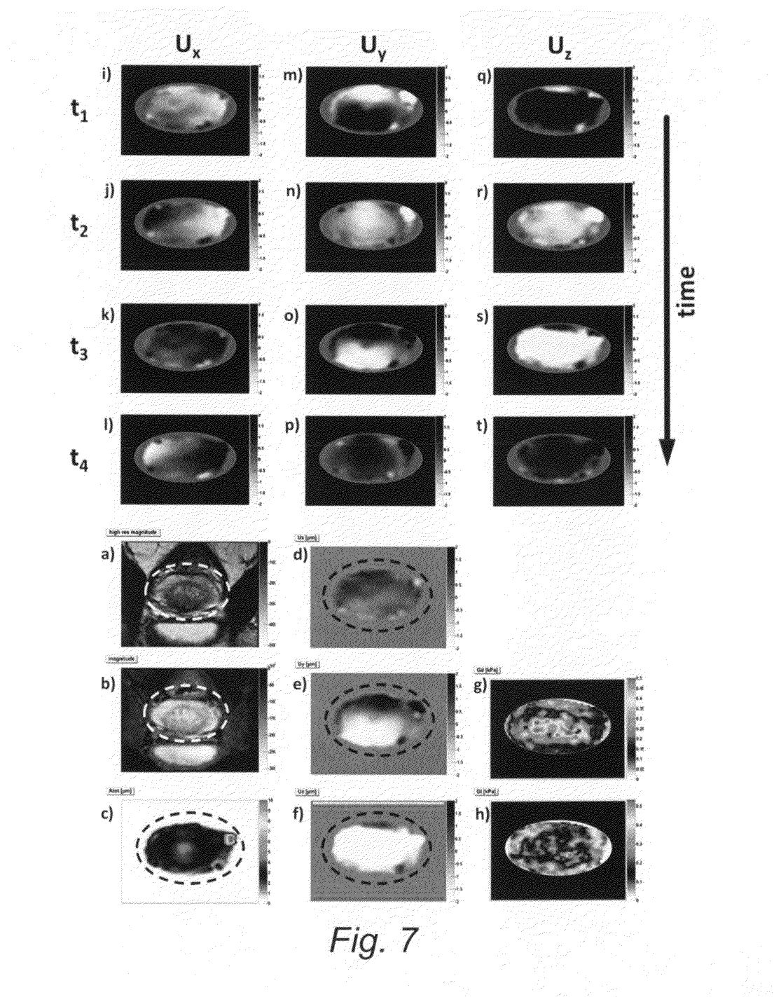

Referring to FIG. 7, sample images acquired by a current embodiment of this invention are shown. The mechanical excitation frequency is about 45 Hz with a maximum amplitude of 2 mm peak-to-peak at the surface of the passive end of the mechanical exciter. Higher frequencies may improve the resolution of the elasticity images obtained. Our hydraulic system is capable of excitation frequencies ranging from 0 Hz to 400 Hz. A typical high resolution T₂ weighted image of a healthy volunteer is shown in 7a where the prostate is marked by a dashed line. The MRE scan protocol magnitude image is shown in 7b. In the following images the tissue surrounding the prostate is suppressed by image post processing software for improved visualization. In conventional MRE the mechanical waves are encoded in the phase images. For example, the displacements in the prostate region at one point in time in the x-, y- and z-direction are shown in 7d, 7e and 7f, respectively. The total amplitude of the mechanical waves is shown in 7c which comprises longitudinal and shear mechanical waves. The displacement images at four snap shots in time in the three spatial directions are shown in 7i-7t. From these displacement images, the viscoelasticity properties can be calculated from the wave equation and displayed as images. For example, if a method by Sinkus et al. (in “Viscoelastic shear properties of in vivo breast lesions measured by MR elastography” in journal of Magnetic Resonance Imaging volume 23, 2005, pages 159-165.) is used, the dynamic modulus (Gd) image and loss modulus (G1) can be reconstructed as shown in 7g and 7h, respectively. The dynamic modulus is a measure of stiffness or elasticity of the tissue while the loss modulus is a representation of how much of the mechanical energy is dissipated as heat. These or similar images that are reconstructed from the raw displacement images may have clinical significance in terms of detection of abnormal tissue (e.g., cancer), classification, staging and monitoring progression of a disease.

### Details on Passive End of Hydraulic Actuator

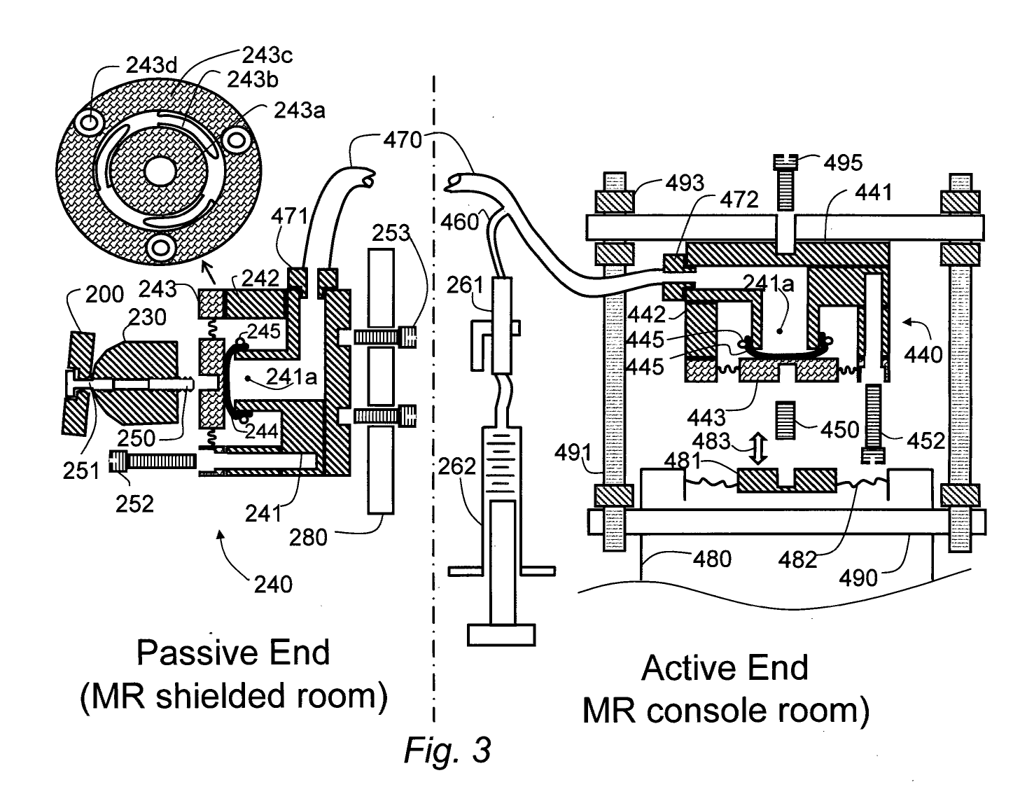

The passive exciter is of cylindrical shape and only a radial cross-sectional view is shown in FIG. 3. Note that the materials utilized in the passive end need to be MR compatible as the passive end is located inside the MR scanner. In the current embodiment the patient-end of the exciter apparatus comprises of the main body 241, spacer ring 242, rubber sheet 244, fastening strings 245, flexure plate 243, transmission element 230, end-point attachment 200 and fasteners 250-252. The semi-rigid tube 470 is connected to the body 241 by a plastic pipe fitting 471. The body 241 is connected to the positioning mechanism 500 by plastic fasteners 253. The rubber sheet 244 is fastened to the main body 241 by wrapping around and tightening nylon strings 245. Silicon RTV is applied at the surfaces where the rubber 244 contacts the main body 241 to ensure a water-tight seal. The fluid that is used in the hydraulic exciter is therefore enclosed in the space 241a, and any increase in pressure from the fluid in the pipe 470 will result in a bulge in the rubber sheet 244. The spacer ring 242 rests between the main body and ensures proper spacing between the rubber 244 and the flexure plate 243. The plate 243a is suspended by the radial flexure springs 243b that are connected to the rim 243c. The holes 243d in the rim 243c are used to fasten the flexure plate 243 to the main body 241 by plastic fasteners 252. When fluid pressure in the hydraulic system changes the suspended flexure plate 243a will be pushed out by the bulge in the rubber sheet. The motion of the suspended plate is transferred by the transmission element 230 and the end-point attachment plate 200. For a linear mechanical response, an absolute pressure of about 1-3 atmospheres is necessary in the hydraulic system so that the rubber sheet 244 bulges and stays in contact with the suspended plate 243a during the whole cycle of the displacement cycle.

The stiffness of the radial flexure springs 243b must be chosen to be stiffer than the elasticity of the tissue. In the current embodiment the spring constant was approximately 10³ N/m.

### Details of Positioning Mechanism of Passive End of Hydraulic Actuator

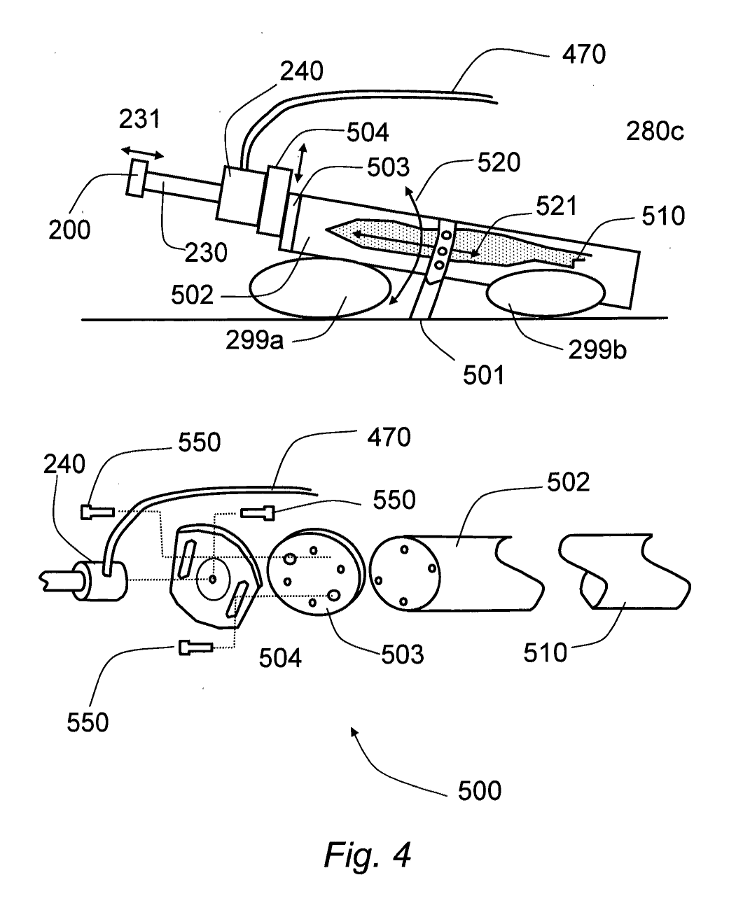

The positioning mechanism is used for aligning the mechanical exciter 240 with respect to the patient 100 on an individual basis and is detailed in FIG. 4. The positioning mechanism 500 comprises a cylindrical enclosure 502 made of a PVC pipe that holds concrete 510. The combination of 502 and 510 act as a rigid weight to which the patient end of the actuator 240 can be attached for efficient transfer of mechanical vibrations to the patient. The concrete filled pipe 502 rests on standard MR safe sandbags 299a and 299b that allow for rough positioning of the exciter 240. A vertical sliding mechanism 503 and 504 are used for easy and more accurate positioning of the exciter 240. The plate with the vertical slots 504a is rigidly attached to the exciter 240 but slides on the plate 503 that is rigidly attached to the cylindrical weight 502.

### Details of the Active End of the Hydraulic Actuator

The active end of the exciter is of cylindrical shape and only a radial cross-sectional view is shown in FIG. 3. Note that the materials utilized in the active end need not necessarily be of non-magnetic type as the active end is located outside the shielded MR room. The active end of the hydraulic actuator is composed of three main components namely the electromechanical exciter 480, active end of the hydraulic exciter 440 and a mechanism that attaches these two components together 490. The electromechanical exciter 480 is a standard commercial electromagnetic exciter (such as the Brüel & Kjer modal exciters series 4824-4828) with the well-known voice-coil configuration. By applying a time varying current to the coils of 480 (not shown), an electromagnetic force will be generated in 480 that is transferred to the moving plate 481, where the induced motion is applied in the vertical direction 483. The active end of the hydraulic exciter is connected to the electromagnetic actuator by the attachment mechanism 480 that consist of a bottom ring, positioning pillars 491, and a top plate 495. The bottom ring is fastened to the commercial electromechanical exciter. The top plate 495 is rigidly attached to the active end of the hydraulic exciter and is vertically positioned with respect to the commercial exciter by the threaded pillars 491 and nuts 493. The hydraulic exciter is rigidly connected to the commercial exciter by a fastener 450. The active end is very similar to the passive end and consists of the same components, namely the main body 441, spacer ring 442, rubber sheet 444, fastening strings 445, flexure plate 443 and fasteners 450, 452. To pressurize the fluid inside the hydraulic system the following additional components are added: a syringe 462, a valve 461 and flexible hose 460. The hose 460 is connected to the main body of the exciter 441 so that the fluid can flow from the syringe in to the space 441a. The pressure in the hydraulic system can be adjusted by opening the valve 461 and adding/removing fluid volume using the syringe 462. The semi-rigid tube 470, which is the same tube that connects to the passive end, is also connected to the body 441 by a pipe fitting 471. The body 441 is connected rigidly fastener to the top plate 492 of the attachment mechanism 490 using fasteners 495.

It is obvious that the active end of the actuator may be located inside the MRI room, as long as it is far away from the magnet such that it does not interfere with the normal operation of the scanner.

### Details of the End-Effector

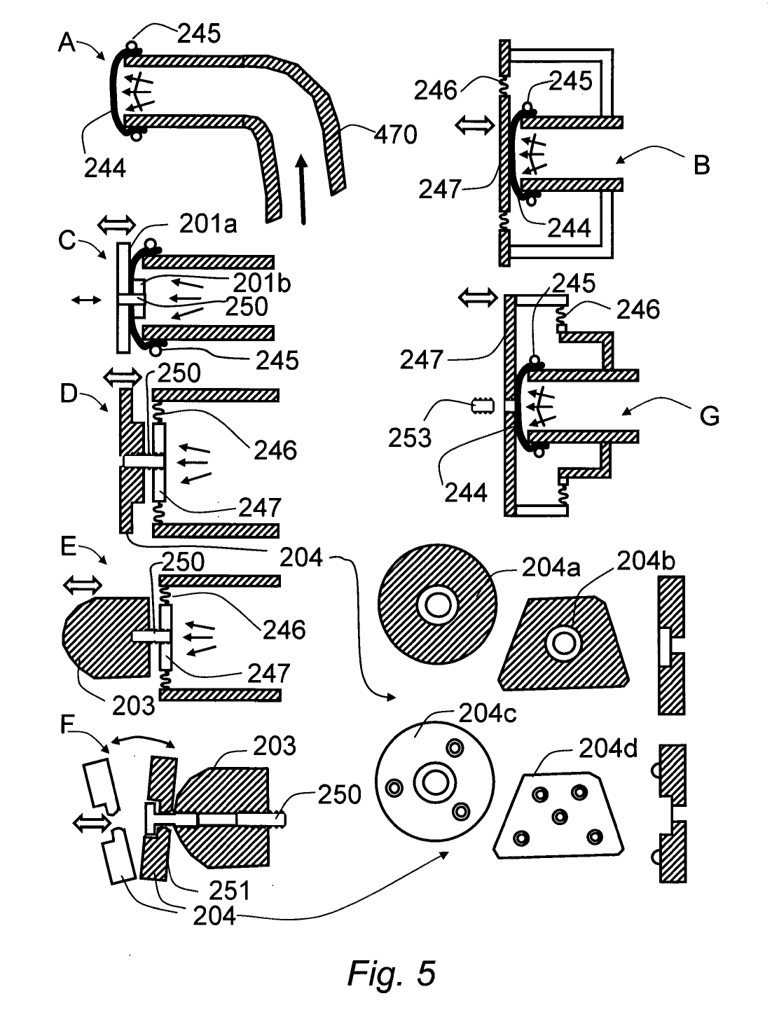

In the current embodiment the passive end of the actuator consists of a compliant rubber sheet 244 that expands and retracts according to the pressure exerted on it by the hydraulic fluid. The displacements of this rubber sheet are transferred to the patient via a suspended plate 243a, transmission element 230 and an end-point attachment 200. Several alternate embodiments are possible, some of which are depicted in FIG. 5. For instance, in one embodiment in FIG. 5A, the rubber sheet may be directly applied to the patient without any additional components. The bulge in the rubber sheets due to the pressurizing fluid will be enough to make proper contact with the tissue of interest. In the alternate embodiment shown in FIG. 5B, the suspended plate can be applied directly to the patient without the transmission element 230 or end point attachment 200. Alternatively, two back-to-back plates 204 and 205 can be used to cover both sides of the rubber sheet as shown in FIG. 5C, in which case the radially shaped displacements can be transformed into parallel displacements that are applied to the patient. In a further alternative embodiment shown in FIG. 5D, the rubber sheet 244 can be eliminated and replaced with radial flexural springs 246 connected to a suspended disk 247 that moves horizontally in response to a change in pressure of the hydraulic fluid. An end point attachment 203 can also be attached using a fastener 250, where the shape of the end point attachment can be shaped as a disc 204a, trapezoidal 204b, knobbed-disc 204c, or knobbed-trapezoid 204d. The shape of the end point attachment can be tailored to the shape of the perineum. The knobbed features on the endpoint attachment may induce additional shear components into the tissue of interest. In another alternate embodiment a piston type of actuator apparatus that comprises of a piston body 210, plunger 211, spring 212 and push rod 213, as shown in FIG. 5H. A pressure fluctuation in the hydraulic fluid that enters the piston body 210 would displace the plunger 211 and pushrod 213. The parts of the pushrod that extends out of the piston is coupled to the tissue, and thus transfers the pressure fluctuations of the hydraulic fluid in 470 to the tissue of interest (here the perineal region of the patient 103). Note that without a spring preload, the cylinder configuration will have an asymmetrical response with limited acceleration when it moves away from the tissue.

Sensors may be added to the endpoint attachment (or elsewhere in the hydraulic system) to detect whether a proper coupling between the patient and the endpoint attachment exists. For example, an optical fibre can be mounted on the endpoint attachment to detect the distance between the patient and the end-effector 200 and inform the operator if the passive end is properly positioned with respect to the patient. This sensor may also operate in on/off mode as a contact sensor. Alternatively, in another embodiment, the pressure in the pressurized hydraulic system may be monitored directly via a sensor to determine if proper contact exists. This sensor may produce and output signal corresponding to the pressure exerted by the end effector on the patient.

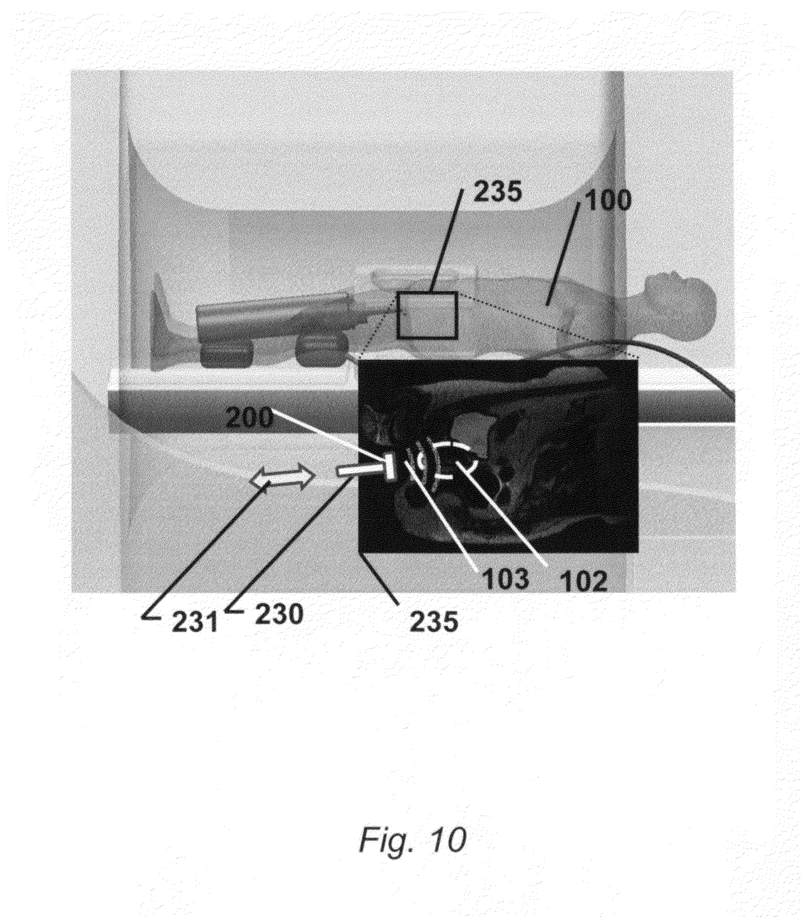

An additional survey scan may be required to determine whether the positioning of the transmission element 230 and the coupling surface 200 is at the correct height relative to the patient's perineum 103. This is illustrated in FIG. 10, where a sagittal scan of the prostate area 235 of the patient 100 is illustrated as a T2 weighted sagittal MRI scan 235. It can be seen that the imprint of the coupling surface 200 on the perineum 103 can be seen in the image. Accurate positioning for MRE using the sagittal survey scan may reduce the need for re-adjustments, as the images may be used to adjust the height of the transmission element and coupling surface 200. For example, if the image of the indentation 103 is seen to be too posterior relative to the prostate 102 (tto low in the supine patient position illustrated), then an MRI technician may adjust the position of the exciter 230 to move the coupling surface 200 anteriorly (lift in the supine patient position illustrated). Alternatively, if the survey scan shows that the coupling surface 200 is too high and presses against the pubic arch, the technician may lower or move the surface 200 posteriorly. It is also possible to quantify such desired motion by locating on the MRI survey scan the location of the coupling surface and the prostate and measuring the offset between the two. Standard techniques for finding the posterior or anterior part of the prostate to produce a mid-line in the sagittal plane can be used to determine the prostate location.

Sensors may be added to the endpoint attachment (or elsewhere in the hydraulic system) to detect the waveform of the vibration. This signal may be used in a closed loop control system to correct for any deviations from the desired vibration waveform.

### Pressurizing the Hydraulic Actuator

To pressurize the hydraulic system, first the hydraulic fluid must be free of air bubbles as any residual air bubbles can distort the mechanical system response (i.e., make the system less linear). First, the two ends of the hydraulic actuator, namely the passive end 240 and active end 440, are immersed in a bath of the hydraulic fluid (here, tap water) so that it enters the spaces 241a and 441a. To facilitate the removal of air bubbles that may be trapped in the corners and dead spaces of 241a and 441a, the actuators can be sonicated in an ultrasound bath or simply tapped manually a few times. Care must be taken so that the syringe pressurizing system 460, 461 and 462 are also free of air bubbles. To fill up the pipe 470, the hydraulic fluid is poured into it while the two ends of the pipe are held at about the same height. Air bubbles are driven out by tapping the pipe 470 starting from the bottom (middle of) the pipe and slowly moving upwards while continuing with tapping action. Since air is lighter that fluid in general, the air bubbles naturally move upwards in a fluid and escape to the atmosphere. While the two ends of the pipe are held at the same height, additional fluid is added until it overflows. Now, the two ends of the pipe 470 are immersed in the bath of hydraulic fluid and the two ends are connected to the actuators 240 and 440. To remove any remaining air bubbles, the active end of the actuator with the syringe pressurizing system is held up at a higher elevation than the passive end 240. Again, by tapping the lower end and moving upwards along the pipe any trapped air bubbles will move to a higher elevation towards the active end of the actuator 440 where they can be extracted from the syringe pressurizing system 462. The very last air bubbles can be removed by inducing some flow in the space 441a in the main body of the active end of the exciter. This can be achieved by adding and removing fluid a few times by the syringe 462. Once the air bubbles are removed, the hydraulic system can be pressurized by adding some fluid volume via the syringe 462 and closing the valve a 461. Pressurising the system is important for achieving high accelerations in both directions of motion of the mechanical vibration source 480.

### Alternate Positioning Mechanisms of Passive End of Hydraulic Actuator

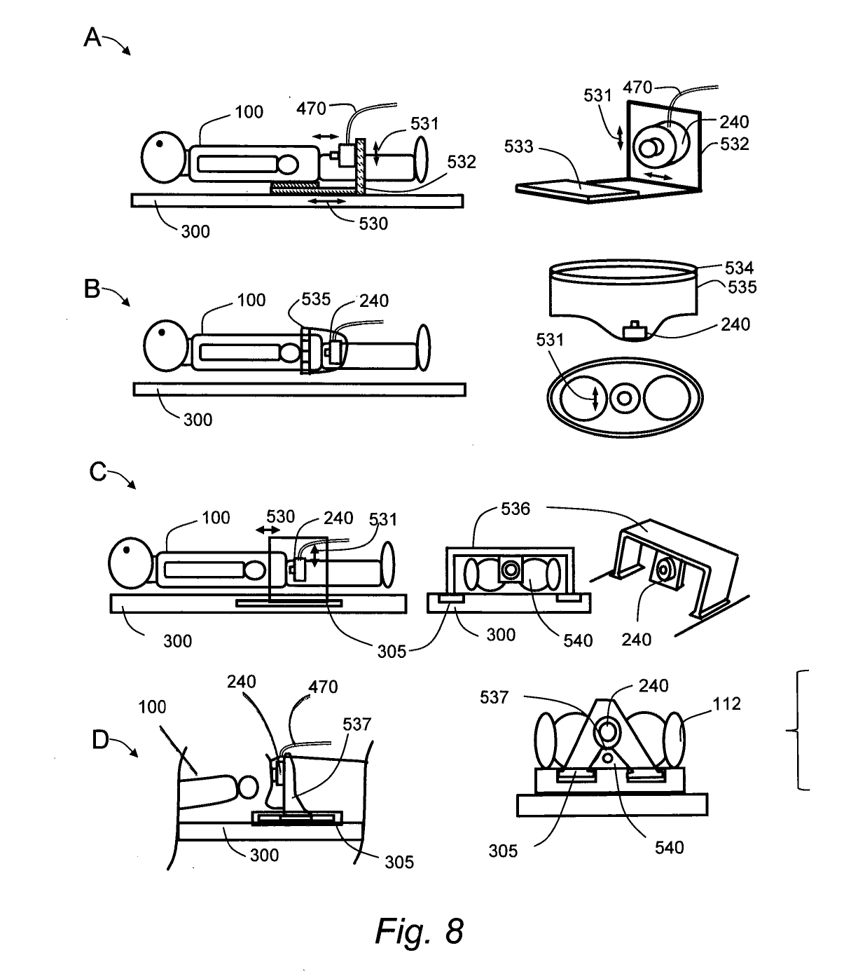

A few alternate embodiments of the apparatus for positioning of the passive actuator 240 with respect to the patient are shown in FIG. 8. For instance, in the alternate embodiment depicted in FIG. 8A, the passive actuator 240 is pushed against the perineum 103 of the patient 100 via an L-shaped mount 535. The mount 532 rests on the MR table 300 and is supported in place by the weight of the patient 100. The motion stages 533 allow the manual fine tuning the position of the passive driver 240 in the anterior-posterior (A-P) direction 531 and also in the superior-inferior (S-I) direction 530. In another embodiment of FIG. 8A the motion mounts 533 may be motorized to allow remote and/or automatic positioning of the passive driver 240. Another possibility for positioning the passive driver 240 with respect to the patient 100 is to attached the driver into a “wearable” shorts 534 that can be positioned in the A-P direction 531. This is shown in FIG. 8B. The shorts 535 are worn by the patient and they are fastened in place by the belt 535. To ensure that the driver stays in contact with the perineal region 103 of the patient, a mechanism (such as spring loaded mount) may be used to push the driver 240 towards the superior direction.

It may be useful to allow access to the perineal region 103 of the patient for example for an interventional procedure during the MRE scan. Also, having access to the rectum 105 would allow the use of an endo-rectal RF coil which would dramatically boost the MR signal for conventional MR and MRE imaging. Therefore, an E-shaped mount 536 can be envisioned (FIG. 8C) that positions the passive driver 240 with respect to the patient and can slide on the standard slots 305 of the MR table 300. This embodiment would allow access to the abdomen of the patient 540 which may become important for an interventional procedure or use of an endo-rectal RF coil. The mount 530 would also allow positioning of the passive actuator 240 in the A-P 531 and also left-right (L-R) 529 direction. In yet another embodiment shown in FIG. 8D, which is a variation of FIG. 8A, the V-shaped mount 537 can be used to position the passive driver 240. The V-shaped mount slides either in the standard slides 305 of the MR table 300 or have its own slides that would fit between the legs 112 of the patient. Again, the mount would allow positioning in A-P, L-R and S-I direction, and may be spring loaded to exert a pressure on the perineum 103 for good mechanical coupling. This embodiment also allows access to the perineum and rectum if needed. In all of the embodiments shown in FIG. 8, markings on the exciter positing devices can be added in order to accurately determine the adjustment of the exciter 200 relative to the perineum 103, i.e., if the survey scan shows that the coupler 200 is 10 mm too high relative to the prostate then an MRI technician can easily adjust manually or remotely the holder mechanism of the exciter 200 to lower the exciter by 10 mm. With an appropriate design for the adjustment mechanism, the patient himself can perform this adjustment while in the MR scanner magnet, without necessitating the movement of the table outside of the scanner magnet and back in for such an adjustment to take place.

In all the embodiment in FIG. 8 the patient 100 is shown to be in supine position (facing up on the MR table 300) but it should be obvious that the patient can be also in prone position (facing down on the MR table 300) with the same type of embodiments shown in FIG. 8.

### Mechanical Safety Mechanisms

It may be useful to have a safety mechanism (not shown) in the hydraulic transmission or the vibration source to disconnect the patient form the exciter in case of a system malfunction. This mechanism may be implemented in several ways. For example, the pressure in the pressurized hydraulic system may be removed (by draining the hydraulic fluid) to effectively stop the transmission of mechanical waves from the vibration source. Alternatively, a mechanical fuse may be implemented to physically retract the passive end of the exciter from the patient.

### Filter

The mechanical exciter, which may include the hydraulic system, may have mechanisms to filter out undesired vibration waveforms. This mechanism may be implemented at the vibration source, passive or active end of the hydraulic system. A typical filter mechanism is a mass-spring-damper system, with resonant frequency selected by the square root of the spring stiffness divided by the mass.

### Mechanism for Attaching Pressurised Pipes

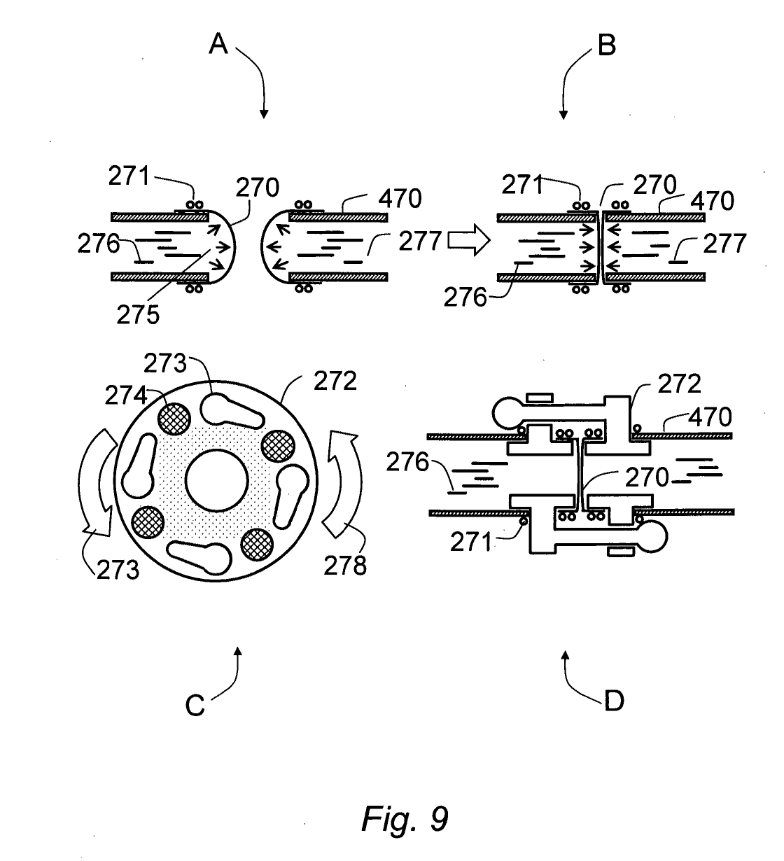

It may be advantages to be able to quickly connect and disconnect the hydraulic transmission pipe/hose without the need to drain/fill the hydraulic fluid and go through a de-gassing process (i.e., remove air bubbles). This idea is illustrated in FIGS. 9A and B. where the ends of two hydraulic transmission pipes 470 (proximal 276 and distal 277 pipes) are shown. Both ends are capped with a rubber sheet 270 that is fastened by a string 271 to the pipes 246 and 277. Any pressure fluctuations in the pipes would result in a bulging/movement of the rubber sheet 270. Both pipe/hoses 2476 and 277 are filled with hydraulic fluid, air bubbles removed and then pressurized. When the two pipes 276 and 277 are separated from each other the pressurized hydraulic fluid 275 forces the rubber sheets to budge outwards. Once the proximal and distal pipes are pressed against each other as shown in FIG. 9B, the rubber sheets contact each other and flatten out (assuming the proximal and distal pipes are pressurized to the same pressure). Now, any pressure fluctuation in the proximal pipe would result in a pressure fluctuation in the distal pipe. This in effect could enable transmission of pressure fluctuations from in the proximal pipe to be linearly transmitted to the distal pipe.

In one possible embodiment, a quick connect/disconnect mechanism can be constructed such as the one shown in FIGS. 9C and D. This mechanism consists of two disc-shaped components namely fastening rings 272 which have several slots 273 and locking inserts 274. One end of each fastening ring 272 is capped with a rubber sheet 270 that may be fastened in place via a string 271. The other end of the fastening ring 272 is shaped such that a hose or semi-rigid pipe 470 can be connected to it which may be fastened in place via strings 271. The two fastening rings 272 can be connected to each other by pressing them against each other while aligning the locking inserts 274 and slots 273 and twisting the ring in the counter clockwise direction 279. To disconnect the fastening ring may be twisted in the clockwise direction 278. An illustration of the connected and locked fastening rings 272 is shown in FIG. 9D where any fluctuations in the fluid pressure in the proximal pipe 276 transfers to the distal pipe 277.

We claim:

1) A method for applying vibrations for generating a magnetic resonance elastography scan of a subject comprising, positioning the end-effector of a mechanical exciter against the perineum of said subject and applying mechanical vibrations generated by said mechanical exciter to said end-effector.

2) A method for applying vibrations for generating a magnetic resonance elastography scan of a subject comprising, positioning the end-effector of a mechanical exciter against the perineum of said subject, inspecting a survey scan image which includes both the prostate and the end-effector, repositioning said end-effector against the perineum based on inspection of said image, and applying mechanical vibrations generated by said mechanical exciter to said end-effector.

3) A method as in claim 1, wherein the mechanical vibrations are transmitted from a remote mechanical vibration source.

4) A method as in claim 1, wherein the mechanical vibrations are transmitted from a remote mechanical vibration source through a hydraulic transmission.

5) A method as in claim 2, wherein the mechanical vibrations are transmitted from a remote mechanical vibration source.

6) A method as in claim 2, wherein the mechanical vibrations are transmitted from a remote mechanical vibration source through a hydraulic transmission.

7) An apparatus for applying vibrations for generating a magnetic resonance elastography scan comprising, a mechanical exciter, means to adjustably couple said mechanical exciter to an end-effector positioned against the perineum of a patient, and means to adjustably couple said mechanical exciter to the scanning table.

8) An apparatus as in claim 7, wherein the mechanical exciter includes a voice coil actuator.

9) An apparatus as in claim 7, wherein the mechanical exciter includes a piezoelectric actuator.

10) An apparatus as described in claim 7, wherein the mechanical exciter includes a mechanical vibration source remote from the patient and a transmission.

11) An apparatus as described in claim 7, wherein the mechanical exciter includes a mechanical vibration source remote from the patient and a sealed, pressurized hydraulic transmission.

12) An apparatus as described in claim 7, wherein the mechanical exciter includes a mechanical vibration filter.

13) An apparatus as in claim 11, wherein a safety valve is used to decouple the transmission from the mechanical vibration source.

14) An apparatus as in claim 11, wherein the sealed pressurized hydraulic transmission includes multiple sealed pressurized segments.

15) An apparatus as in claim 7, wherein the end effector is removable and selectable among several end-effectors of different shapes for better coupling to the perineum.

16) An apparatus as in claim 7, wherein the means to adjustably couple the mechanical exciter to the table allows the simultaneous insertion of an endo-rectal coil.

17) An apparatus as in claim 7, wherein the end-effector includes a sensor to determine whether proper contact to the perineum exists.

18) An apparatus as in claim 7, wherein the end-effector includes a vibration sensor to determine proper vibrations are transmitted.

19) An apparatus for applying vibrations for generating a magnetic resonance elastography scan comprising, a mechanical exciter, means to adjustably couple said mechanical exciter to an end-effector positioned against the perineum of a patient, and means to adjustably couple said mechanical exciter to wearable shorts.
